(19) United States
(12) Patent Application Publication
(10) Pub. No.: US 2011/0127405 A1
(43) Pub. Date: Jun. 2, 2011

(54) OPTICAL STIMULATION OF PHOTOSENSITIZED CELLS

(76) Inventors: Nir Grossman, London (GB); Patrick Degenaar, Newcastle upon Tyne (GB); Konstantin Nikolic, London (GB); Mark Neil, Botley (GB); Gordon Kennedy, London (GB); Vincent Poher, Guines (FR)

(21) Appl. No.: 12/992,963

(22) PCT Filed: May 18, 2009
(86) PCT No.: PCT/EP09/55992

§ 371 Date: Feb. 5, 2011

(30) Foreign Application Priority Data
May 16, 2008 (GB) 0808965.8

(51) Int. Cl. G01J 1/16; A61N 5/06; G02B 21/06

(52) U.S. Cl. 250/201.1; 250/492.1; 359/385

(57) ABSTRACT

This invention describes a device for optical stimulation of cells and other biological structures. It has an ability to target multiple cells and/or multiple sub-cellular targets. The stimulation optical pattern on each cell can be independently controlled with individual frequencies. The light sensitivity of the cells can be imparted as a result of genetic expression of surface and/or subsurface proteins, chemical modification of existing proteins, or via the release of caged entities which in turn act to stimulate the cell through chemical means. The embodiment is capable of functioning on neurons but can also be used for other cells. It can perform optimal stimulation with sub-cellular resolutions, record the activity of the targeted cells and perform processing to ensure calibration.

### FIELD OF INVENTION

This invention relates to methods and apparatus to optically stimulate light sensitized cells.

### BACKGROUND TO THE INVENTION

Over the years we have gained considerable insight into the functioning of neurons since the original discoveries in 1952. Patch clamping has been an effective tool to investigate the biochemical factors involved in neural signalling. More recently the emergence of multi-electrode array (MEA) technology has provided researchers with multi site recording and even stimulation capabilities. MEA technology is used today extensively to study the dynamic of interactions within neuron networks, synaptic plasticity visual perception and effects of pharmacological compounds and putative therapeutics.

Although an MEA provides a good recording means and has advantages over patch clamp in that multiple stimulation and recording sites are accessible, MEA's suffer from major drawbacks. There is a short dead time between the stimulation pulse and the ability to record, as the large pulse will saturate the sensitive pre-amplifiers. The stimulation spatial resolution is limited by propagation of the electrical pulse through the solution and thus all the neurons within the vicinity of the stimulation electrode are potentially excited. Additionally, the position of the stimulation points in respect to the sample are fixed, once the sample is laid down on the substrate with the electrodes. In general, a specific electrode on the microelectrode array can either be wired to a stimulating oscillator or a recording preamplifier, not both. Finally, it is only possible to electrically excite action potentials, not inhibit them.

Recent advances in biochemistry that enable the optical excitation of cells have started a new era in the study of neural physiology, especially at the network level. However, the photo-stimulation concept is not actually new. It was first demonstrated in 1971 by Richard Fork who used a high power laser to stimulate action potentials in the abdominal ganglion of the marine mollus Aplysia Californica. Since then scientists have exploited developments in nanotechnology and genomics to photosensitize cells. These sensitization methods can be divided into three categories of useful techniques.

The first category is photolysis of caged neurotransmitters, which was also the first modern photo-stimulation technique. Neurotransmitters, rendered inactive with covalently bonded blocking ligands are released into the solution around the neurons. The blocking moiety bonds with the neurotransmitters are then broken with the use of UV light. The result is a great localized increase in the neurotransmitter concentration in the vicinity of the light spot, which can excite a neuron cell. Due to its wide expression in the central nervous system (CNS) neurons, caged-glutamate is the most commonly used caged neurotransmitter. The uncaging process has a very good temporal and spatial resolution (approximately 3 ms and 5 μm, respectively)—the later mainly limited by the diffusion of the uncaged molecules. The release of such caged molecules can be used not only to stimulate neuron cells, but also to stimulate non-neuronal cells and can be useful for potential drug screening.

The second category involves the incorporation of photo-switch-linked ion channels on the cell membrane. These can come in the form of modified ion-channels with light gated opening mechanisms. The modification method can be fully genetic, fully chemical, or a mix of the two. Presently the most promising channels for prosthetic applications are genetically incorporated opsin channels and pumps such as channelrhodopsin2 (ChR2) and halorhodopsin (NgHR) (Banghart, Borges et al. 2004; Zhang, Wang et al. 2007). These can depolarize and hyperpolarize cells respectively. As they are genetically incorporated in cells, they will be continuously produced by the cellular machinery circumventing any decay over time. Delivery methods include plasmid and viral (e.g. Adeno Associated and lenti virii) transfection. Existing ion channels and receptors can be genetically modified to receive photo-switchable arms which can act to open or close the active site. Present examples include hyperpolarizing shaker channels and depolarizing modified glutamate channels. Presently, while it is conceivable to chemically induce photosensitivity into existing membrane receptors, no such system exists without genetic manipulation. Such a process would involve matching a targeted amino acid sequence to bind to the outside of an anchor point, and the use of a photo-isomerisable arm which can insert or retract an agonist into the active core.

The third category involves the use of photo-switch activated amplification cascades linked to a particular ion channel. Such systems exist naturally and are the basis of all invertebrate and vertebrate (including human) sight, where some form of opsin protein (e.g. rhodopsin in humans and metarhodopsin in flies) is the active photo-switch, which connects to a G-protein cascade which acts to depolarize or hyperpolarize specific cells. Another example is Melanopsin which triggers a cascade that is believed to activate TRP ion channels, resulting in depolarisation of retinal ganglion cells. Both the two opsin photo-switches described exist in the membrane, but it is conceivable that other alternative forms of photo-switching linked cascade could be developed around alternative routes to opsin proteins. These cascades would need to be genetically engineered into the cells such as through viral (e.g. lentivirus), lipid or other transfection methods.

Common to all of these optical methods is the requirement to provide sufficient light stimulus of the appropriate wavelength (photon energy). The most developed technique, the use of ChR2, requires the illumination of a pulse of light having energy between 10 pJ-10 nJ depending on expression levels and method of stimulation in order to generate an action potential. We have shown previously more in-depth models of such stimulation (Nikolic, Degenaar et al. 2006). In the case of caged molecule release, similar energies are required, albeit in the UV region of the spectrum (V. Poher, N. Grossman et al. 2008). Thus, given such large intensity requirements, people have previously employed high power illumination sources such as Xenon/Mercury lamps, lasers or high power light emitting diodes (LEDs) in conjunction with a microscope or optical fibre setup. Our group demonstrated the use of arrays of high power GaN LED arrays to achieve patterned illumination with sufficient intensity (V. Poher, N. Grossman et al. 2008). It may be possible in the future, with modified photosensitization agents to have lower thresholds, in such a situation light emissive systems with lower illuminations such as OLED arrays could be used.

The advantages of such photo-stimulation of cells compared to traditional electrical approaches are many: The light beam can be easily focused to very high spatial resolution using conventional optics, limited only by diffraction. The location of the stimulating beam relative to the neurons can be easily changed, in contrast to the case of fixed electrodes on a microelectrode array. Because the stimulus signal is light, there is no interference between the stimulus signal and the recording electrodes and hence there are no issues with biocompatibility and gradual degradation of the electrode-cell contact. Action potentials can be inhibited with the use of photo-inhibiting agents that are sensitive at different wavelengths to the photo-stimulating agents. Additionally, individual neuron types in a heterogeneous culture can be selectively stimulated by using genetic targeting with different photosensitization agents. For example, ON and OFF pathway cells in a retinal slice could be sensitized with depolarizing and hyperpolarizing agents.

The advent of novel light-gated cation channels in 2003 has allowed for the first time, viable optical stimulation of nerve cells in culture and in-vivo. Since then, there has been an explosion of research to understand and exploit the processes further. What has been missing, however, has been a fully integrated system which can simply be plugged in to a microscope. The commercial availability of such a tool would be of great use to biologists and other interested parties in the field of in-vitro electrophysiology, drug efficacy, plasticity, neural signally, and cell growth, neural computer interfaces We previously presented the basic concept of light stimulation of neurons from GaN LED arrays (V. Poher, N. Grossman et al. 2008), but have not fully presented how a fully functional system may be comprised. Similarly, other groups have proposed alternative schemes based on light sources connected to micro-mirror arrays, bundles of optic fibres and scanning lasers (Bernardinelli, Haeberli et al. 2005). For many reasons these approaches are not optimal.

It is known, for example, from WO/2007/148038, to use the optical stimulation of light sensitized cells in prosthetic devices.

### SUMMARY OF THE INVENTION

The present invention provides a system for optical stimulation of cells, comprising an array of light sources, an optical system for directing light from the sources to a sample location, and control means to control the operation of each of the sources.

The optical system may comprise a plurality of light source lenses, each arranged to direct light from a respective one of the light sources. The light sources may have gaps between them so that only a fraction of the area of the array is light-producing, and the lenses may be arranged to direct the light so that, in the image of the sources on the sample location, a greater fraction of the image area is illuminated with light from the sources. The lenses can therefore be arranged to fill, at least partially, gaps between the light sources.

The optical system may comprise at least one imaging component arranged to form an image of the light sources at the sample position. The optical system may be arranged to cause convergence of the light from the light sources onto an area at the sample which is smaller then the light source array. This can be advantageous, for example because stimulation of cells in specific localities can improve the kinetics of stimulation.

The optical system may be arranged to provide spatial resolution in the image of less than 10 microns, and in some cases of 1 micron or less. The system may also be arranged to achieve an intensity of at least 100 pW per square micron.

The control means may be arranged to control each of the light sources independently. For example the control means may be arranged to control at least one of: the intensity, the frequency of illumination pulses, and the duration of illumination pulses of the light sources. In some cases pulsing of the light from each of the light sources is required, and in some embodiments the pulse length, or the pulse period, is arranged to be less than 1 ms. Our experiments show that reducing pulse width can in some cases improve the kinetics of the photosensitized cells, improve their long term survival, and reduce the overall power consumption of the system.

The system may further comprise sensing means, which may be for example a multi-electrode array or patch clamp electrodes, arranged to sense the response of cells in the sample to the stimulation. The control means may be arranged to receive signals from the sensing means and to control the light sources in response to the signals. For example the control means may control the light sources so as to achieve a desired response in the sample.

The system may comprise adjustable mounting means on which the array of light sources is mounted, and the control means may be arranged to adjust the mounting means to adjust the position of the array. Again this control may be provided on the basis of feedback from sensing means so as to achieve a desired illumination of a sample position.

At least one component of the optical system may also be adjustable, and the control means may be arranged to adjust said at least one component. In some embodiments, the light sources and light source lenses are mounted on a chip on which the optoelectronic control means are also formed.

The present invention further provides a method of calibrating a system according to the invention, the method comprising: measuring the total illumination in an image of the light sources, measuring a variance in illumination intensity over at least a part of the image, and controlling the light sources to achieve a desired variance in illumination intensity over at least a part of the image.

The method may further comprise determining the absolute intensity at at least one point in the image. The light sources may be controlled so that they each provide the same level of illumination.

The present invention further provides a microscope system comprising a light source arranged to illuminate a sample position, an objective lens arrange to image the sample, an optical stimulation system according to the invention, and light directing means arranged to direct light from the array of light sources onto the sample position. The light directing means may be arranged to introduce the light from the array of light sources into the optical path of the microscope on the opposite side of the sample position to the objective lens, in a trans-illumination arrangement. Alternatively the light directing means may be arranged to introduce the light from the array of light sources into the optical path of the microscope on the same side of the sample position to the objective lens, in an epi-illumination arrangement. The light from the array of light sources may also be introduced into the light path on the same side of the sample position as phase contrast illumination in the microscope, or on the opposite side. The light from the array of light sources may also be introduced into the light path on the same side of the sample position as fluorescent illumination in the microscope, or on the opposite side.

The system may be integrated electronically with electrophysiological equipment. Such integration may take the form of control signals via a computer or other electronic device or directly through a combined controller.

The present invention further provides a method of optically stimulating cells comprising providing a system according to the invention and controlling the light sources to stimulate a sample at the sample location. An image of the light sources may be formed on the sample, so that control of each of the light sources controls the illumination in a different part of the image. Light from a plurality of the light sources may be imaged onto a single cell, for example a neuron, such that sub-cellular components can be independently illuminated.

In one aspect, the invention provides a device for multi-site stimulation of biological cells or tissues based on light. It may be designed to work with existing recording techniques, such as patch clamp electrophysiology, extracellular recording such as microelectrodes array, cellular calcium imaging, or other fluorescently linked metabolic imaging methods. The system may consist of an array of miniature light sources such as light-emitting-diodes (LED)s that is imaged on to the sample. It may have optics for integrating the micro-light sources into a microscope or incubator. The stimulating pattern (amplitude, pulse width and repetition rate, wavelength) of each stimulating spot may be independently tuneable in real time. The actual position of the light dot array can be easily finely tuned in the focal plane by moving the platform with the sample on the microscope. The invention can also provide a method for a closed-loop control where the responses of the cells are fed back to the system and used to tune the stimulating light pattern, a method to determine the photon flux per cell, a method for calibration and a method to cool the micro-light sources for higher efficiency.

In some embodiments the invention can be incorporated into both upright and inverted microscopes, in both epi- and trans-illumination configuration for each. We describe in detail the incorporation of the device for the inverted microscope case, but it is effectively the same as for the upright.

### Light Source

Embodiments of the invention may incorporate the use of an array of light emissive elements with which to stimulate the neuron cells and other biological structures. As the present biological technology requires high brightness, we can use Gallium Nitride LEDs, which have an advantage in being easier to tune. Other LED sources such as from organic semiconducting polymers (OLED) could potentially be used if they could achieve sufficient irradiance. In the future modification to the photosensitization agents could reduce the illumination requirement, thus rendering such sources viable. Additionally, a closely related embodiment would be the use of vertical cavity surface emitting lasers (VCSEL).

### LED Driving Electronics

The light source for embodiments of this invention may come from bright light emissive diodes, but could conceivably come from VCSEL laser or other light emitting products. However, the circuitry generally follows the same principles. The LEDs can be passively driven via raster scan control, in which case generally digital logic processing controllers such as PICs and FPGA's would be sufficient. Alternatively the LEDs can be individually driven with circuits at each pixel. Given the complexity in control line alignment, if there are more than 384 pixels, the ideal embodiment is for a CMOS control chip which is combined with individual control lines to each LED.

### Light Emitting Device Micro Optics

If the light source comes from a LED's or VCSEL's array, the individual emitters will generally have spacing between them. Thus it can be advantageous to incorporate micro-optical components such as micro-lenses to increase the fill factor. As a result the light sources, e.g. LED's, can be arranged into both square matrix and hexagonal arrays to achieve maximum efficacy. In the case of GaN LEDs, which are presently the optimum light source, the substrate can be transparent, so it is possible to emit from either the top or bottom side. Bottom side emission allows for the top side to be easily bonded to CMOS controller chips. Additionally the layering of the substrate results in some natural diffusion of the light emitted from the LED sources.

### System Optics

The optics of the described invention can facilitate the incorporation of one or more light emissive sources. The use of beam splitters and/or wavelength dependent mirrors can facilitate the addition of multiple light sources to be overlaid over each other. Additionally, lenses may be used to manipulate the light beams from the light emissive arrays, and to focus the combined beams on the sample. In the case of the epi- version of this invention, a modification may be carried out on the microscope filter whereby the output barrier filter is removed from its normal position and a new filtering component is incorporated nearer the camera. As such it is therefore advantageous to combine any image recording system such as a digital camera within this described method.

### Opto-Mechanics

Some embodiments of the invention incorporate opto-mechanical constructs to finely adjust the optical components such as the light emitters and the mirrors to achieve optimum alignment, imaging and light throughput. Additionally, individual optical components to combine the light beams can be inserted, retracted or switched in configuration. These fine adjustments and switching can be performed manually or electromechanically through stepper motors, linear actuators, piezo crystals and other such devices.

### System Electronics and Computer Control

The light emissive system contains many individual elements which require individual control. It is therefore important to have electronic automation which in turn is controlled via suitable control means such as a computer or dedicated electronic controller. The control interface could be through wired high speed serial such as USB, or firewire, parallel interfaces such as GPIB-488, or wireless such as through the 802.11g protocol. The controller will then send signals to the electronics to determine the individual illumination frequencies of individual LED's while monitoring the resulting response from the electrophysiology and/or imaging components.

In addition the controller may have software which generates a user interface to allow the user to determine exactly how much light is falling onto each part of the neuron. This may be correlated to neuron response via additional spike sorting algorithms. Importantly it may also have calibration tools connected to the camera allowing calibration of the light intensity. Additionally a feedback loop can be implemented whereby, when a given neuron response is not being achieved by the neuron, the light intensity and/or pulse duration impinging on the neuron is adjusted.

Preferred embodiments of the present invention will now be described by way of example only with reference to the accompanying drawings.

### BRIEF DESCRIPTION OF THE DRAWINGS

FIG. 1A is a diagram of a system for optical stimulation of cells according to a first embodiment of the invention;

FIG. 1B is a diagram of a system for optical stimulation of cells according to a second embodiment of the invention;

FIG. 2 is a diagram of a system for optical stimulation of cells according to a third embodiment of the invention;

FIG. 3 is a diagram of a system for optical stimulation of cells according to a fourth embodiment of the invention;

FIG. 4 is a diagram of an optical array suitable for use in the systems of FIGS. 1 to 3;

FIGS. 5A and 5B are diagrams of drive circuits for the optical array of FIG. 4;

FIG. 6 shows images produced with a system according to one embodiment of the invention.

### DETAILED DESCRIPTION OF THE PREFERRED EMBODIMENTS

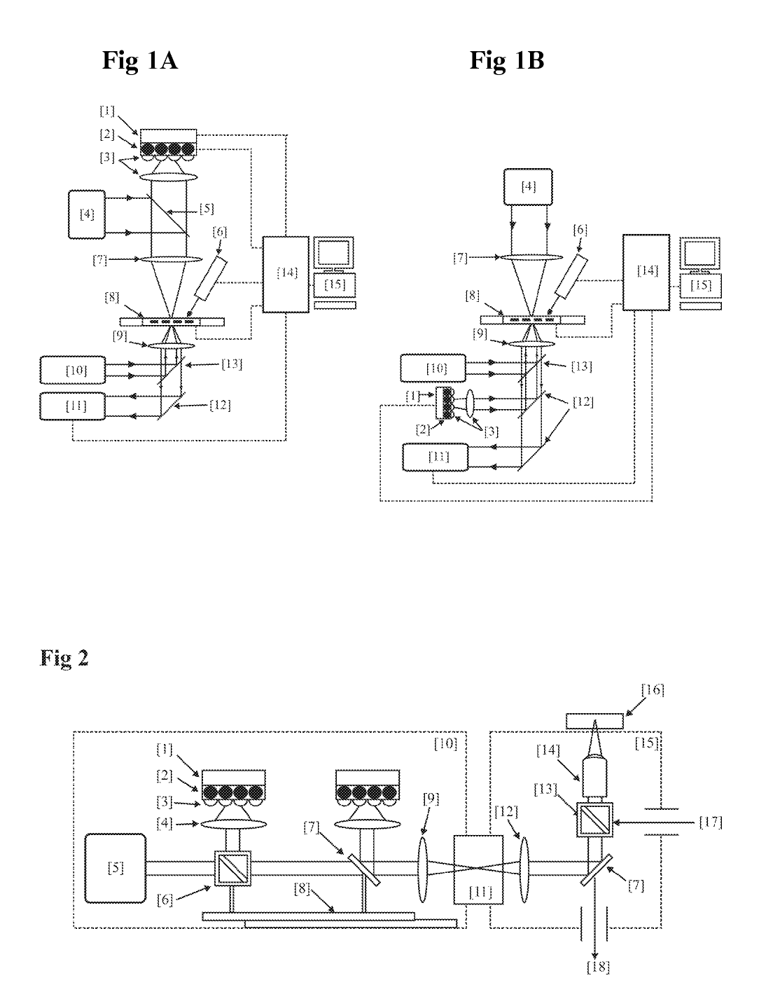

Microscope configuration. In a microscope configuration, the light emitting array can be configured in a trans- or epi-illumination manners. Referring to FIG. 1, in one embodiment arranged for trans-illumination, a light source array 2 which includes electronic control circuitry is mounted on an adjustable stage 1 which is arranged to allow fine tuning of the position of the light source array. A lens system 3 comprises an array of lenses each associated with one of the light sources, and a further lens located below the light source array 2 through which light from all of the sources passes downwards. Focusing optics 7 in the form of a further lens 7 are arranged to focus the light from the lens system 3 downwards onto a holder 8 which is arranged to hold sample cells. A microscope objective lens 9 is located below the holder 8 and microscope internal mirrors 12 are arranged to direct light from the objective lens 9 to a microscope eyepiece and camera 11. A fluorescent light source 10 for the microscope is also provided and a fluorescent light filtering unit 13 is located between the objective lens 9 and the internal mirrors 12 to direct fluorescent light from the fluorescent light source 10 up through the objective lens 9 to the underside of the holder 8.

An illumination light source 4 is also provided and a half mirror 5 between the light source lens system 3 and the adjustable optics 7 is arranged to direct light from this illuminating light source 4 down on to the sample cells on the holder 8. A patch clamp electrophysiology unit 6 is arranged to detect the response of the sample cells to the optical stimulation of the stimulating light source 2. A control system comprises a data acquisition unit 14 arranged to receive image data from the camera 11, cell response data from the patch clamp unit 6, data regarding the position of the holder 8, data from the adjustable stage 1 indicative of the position of the light source array 2. The control system is also arranged to control the lights sources in the array 2, being able to switch each of the light sources on and off independently.

In this arrangement, the array of light sources 2 can be imaged using the light source and focusing lenses 3, 7 in a relay arrangement. The light is coupled to the optic path of the microscope with optics such as beam splitter, dichromic mirror or semi-transparent mirror. The position of the array 2 can be fine tuned with the 3D positioning stage 1 either manually with a screwing mechanism or electronically with a piezo or motorized stage under control of the control system 15. In this trans-illumination embodiment the array of light spots is coupled to the optic path of the microscope light source using a beam splitter or semitransparent mirror 5 for example. The second lens 7 is placed after the beam splitter 5 and can be used also as a condenser for the light from microscope lamp 4. In case of a phase-contrast microscopy, a condenser annulus mask is placed near this lens.

Referring to FIG. 1B, in which components corresponding to those in FIG. 1A are indicated by the same reference numerals, in epi-illumination the array 2 is coupled to one of the microscope ports. The array 2 can also share a port with other devices such as camera 11 or epi-fluorescent light source 10. In the case of an array-camera configuration, a beam splitter, dichromic mirror or a semi-transparent mirror 12 is used to couple the array light to the optic path. The emission filter is then placed in front of the camera 11 (instead of in the filter box 13 next to the objective lens) so the light from the array 2 is not filtered out. When the array 2 is sharing a port with epi-fluorescent source 10, the beam splitter 12 is used to couple the array light to the optic path of the epi-fluorescent light. In this case, the excitation and ND filters are placed near the epi-fluorescent light source 10 before the beam splitter so they will not filter out the light from the array 2. A long pass filter removes the blue light from the illuminator in order to image the cells without invoking stimulation.

Enhancement components. Micro-lens on each micro emitter, forming part of the lens system 3, can be used to increase the effective fill factor of the array 2 and hence may be useful in allowing the smaller fill factor. Photonic crystal structures can be used to improve the extraction coefficient of the light from each emitter. Since the radiation efficiency typically decreases with temperature, a cooling system based for example on fan or thermoelectric effect can be introduced to maintain high efficient functioning.

### Other Configurations

Multi-cell and Sub-cellular resolution. The diameter of a single illumination spot generated in the systems of FIGS. 1A and 1B is typically 20 to 50 micrometers. Using a 1:1 magnification, each light spot covers approximately a single cell. This arrangement enables simultaneous patterned stimulation of thousands of cells. Alternatively, each spot of light can be de-magnified by further lenses included in the system so that hundreds of light spots cover a single cell. In this case, stimulation with sub-cellular resolution can be achieved. This can be very useful for fundamental study of neuron functions and single cell computing.

Multiple wavelengths configuration. In some cases it is beneficial to illuminate multiple arrays that have different wavelengths. This can be used for example to trigger both channelrhodopsin (ChR2) and halorhodopsin (NgHR) independently. A multiple array configuration can be realized for example by stacking multiple beam splitters or dichromic mirrors in the optic path and coupling light array per splitter. The splitters or dichromic mirrors can be designed to reflect just one specific wavelength of one of the light source arrays, and not the wavelength or wavelengths from the other array or arrays. In this way, the optics from a single array is not affecting the light from other arrays with different wavelength. Referring to FIG. 2, in one such embodiment which is an epi illumination system, a microscope comprises a housing 15 having a fluorescent light input 17 in one side, an output 18 to an eyepiece and a port 11, which can be a side, rear, back or bottom port. A sample holder 16 is provided in this embodiment on top of the housing 15 and an objective lens 14 is located beneath the holder 16. A fluorescent light filter 13 is arranged to direct light from the fluorescent light source 17 into the light path of the microscope and to filter fluorescent light from the output. A half mirror 7 between the filtering unit 13 and the output 18 splits the light path between the output 18 and the port 11. Microscope port optics 12 are provided adjacent to the port 11. An LED stimulation unit comprises a housing 10 with two stimulating light source arrays 2 each with associated optics 3, 4 and mounted on adjustable stages 1. The two arrays are arranged to generate light of different respective wavelengths. A lens 9 is provided adjacent the port connector 11 at one end of the unit and a camera unit 5 at the other end. Half mirrors 6, 7, one of which 6 forms part of a light filtering unit, are arranged to introduce images of the light source arrays 2 into the light path of the system between the camera 5 and the port 11. The half mirrors 6, 7 are mounted on an adjustable stage 8 which enables the optical focus of the system to be optimized.

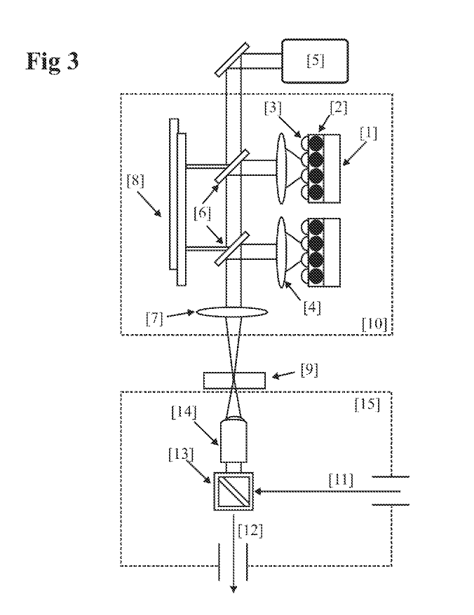

Referring to FIG. 3, a top illumination multi-wavelength system is similar to that of FIG. 2. The microscope 15 is set up in the same way, and corresponding components are indicated by the same reference numerals, except that the LED stimulation unit is arranged above the sample holder 9. The stimulation unit is essentially the same as that of FIG. 2, with corresponding components indicated by the same reference numerals except that the fluorescent light input, still through the side port of the microscope, is indicated as 11.

Stand-along configuration. In another configuration the cells or tissue is placed directly above the light emitter array. This approach can be useful for long stimulation experiments whereby the cells are grown for long periods of time, for example in an incubator. In this configuration, all the electronics and the emitter array are packaged in a manner that keeps them safe from humidity or water drops. The light from the emitters is coupled out through a transparent window in the package. In that case, the cells are cultured on a cover slip, or a special stimulating chip that have a mini ring to contain cell medium with components to perfuse medium and maintain temperature. There may also be a cover to provide additional protection against evaporation of cell medium. In this case the packaging box of the stimulation platform fits exactly above the light emitters.

In-vivo configuration. The same platform can be used for in-vivo photo-stimulation. The advantage of this technique is that it enables multi-site in-vivo stimulation with micrometer spatial resolution. Such a configuration can be used for imaging live animals in a modified microscope setup. The emitter array is imaged on the body or organ using micro lenses or lenses relay. Alternatively long working distance lenses can be used to focus the light from a distance onto the target area, e.g. the brain. In this case a clamping system is required to keep the LEDs stationary relative to the target area. The control and driving circuitry can be kept separate from the light emitter for better compatibility.

### Light Sources

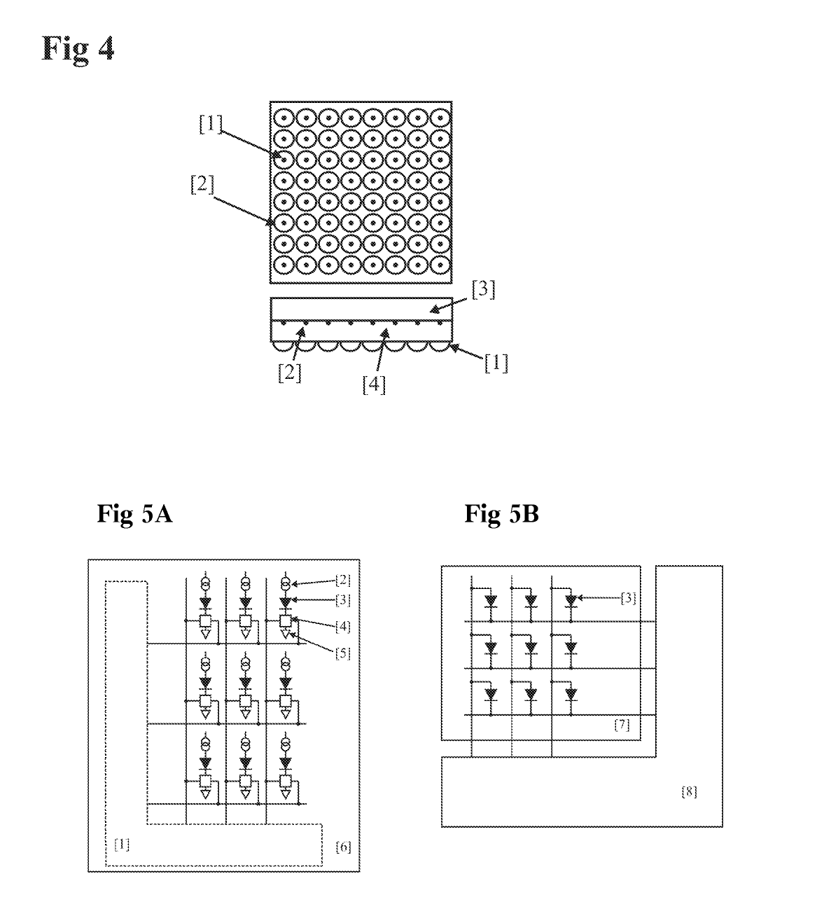

Referring to FIG. 4, the light source 2 in each of the embodiments described may be an array of micrometer high-power light emitters in the visual and UV region. One example is a micro-LED array based on Nitride semiconductors such as Aluminium-Nitride (AlN), Gallium-Nitride (GaN), Indium-Nitride (InN) and their alloys. Using these semiconductors LEDs in the entire visible range and deep UV wavelengths can be potentially realized. Since the band structure of these semiconductors has a direct band gap across the entire alloy range, it allows the nitride-based LEDs to exhibit high quantum efficiencies that together with their narrow spectral emission enables high brightness which is essential for the current application. The LEDs 1 are arranged in an array on a chip, supported on a substrate 4, with a micro-optical component such as a micro-lens 2 over each of them. Control electronics 3 for controlling the LEDs 1 is provided, for example on the back of the substrate 4 in a flip chip arrangement.

For example, a 64 by 64 matrix-addressable and a 120×1 stripe addressable micro-pixelated nitride based light emitting diodes that have been developed under the RCUK Basic Technology Project, and their applications have been previously demonstrated (V. Poher, N. Grossman et al. 2008). It can have a matrix addressed or flip-chip configuration. Another example is an array of micro lasers such as vertical-cavity-surface-emitting-lasers (VCSELs), or array of organic light emitting diodes (OLEDs).

### LED Driving Electronics

The light source for some embodiments of this invention comprises an array or bright light emissive diodes, but can alternatively come from VCSEL laser or other light emitting products. However, the circuitry generally follows the same principles. The light emission can be controlled independently for each of the array of light sources by applying a specific voltage and allowing the device to determine the current. Alternatively, it is possible to drive the device at a specific current and allowing the device to determine the voltage. The difference is subtle but important. As the generated photons result from the current injection, generally a much more linear and stable light emission characteristic is achieved by operating in current source mode rather than voltage source.

The total number of photons hitting the target is related to the integral of the intensity with time. Thus the effective intensity can be varied by varying either the intensity or the illumination pulse time, or both. In most cases the most convenient implementation is to pulse the light at the maximum intensity and simply vary the duration of the pulse.

The light emitters in one embodiment are passively driven via raster scan control, as can be seen in FIG. 5(B). In this case, the emitters are arranged on a GaN LED chip 7 each having a respective control wire 3 enabling it to be controlled, with minimum requirement for control electronics built in. Instead a simple raster of the rows is required while turning on specific desired columns. The raster control can be performed via digital or analog logic components making up control electronics 8, but generally programmable digital logic such as an FPGA is the most cost-effective. A circuit board can house individual power components and provide discrete current sources if required.

The passive array is simple to implement but has limitations in that only one row can be illuminated at a time which reduces the overall potential integral illumination intensity when scanning the whole array. For this reason, active control, whereby each light source has its own continuous control is optimum. However, most LED and VCSEL configuration are limited in terms of the electronic components which can be integrated on the chip. Thus, external controller chips need to be used. However, the number of input lines which can be addressed from external chips is limited. Thus the optimum configuration is for a CMOS chip to be sandwiched with the light emitting chip, whereby individual control lines for each Light emitter can be addressed through a matrix of connection points.

Such an active array, as shown in FIG. 5(A) which comprises an active driver chip 6 which is flip-chip bonded to the GaN LED chip. The arrangement has space to allow for individual control electronics per pixel, as well as an information stream decoder and line and column control electronics 1. The simplest arrangement is for a memory unit such as a flip flop 4 at each pixel to be connected to a gate which allows current through the light emitter. In this case a raster or asynchronous address event system can be used to turn individual pixels on and off. Decoder circuitry in the side of the chip can be used to decode desired oscillation frequencies into required address events or rastered updates of status. Other configurations include oscillator circuitry with or instead of the memory unit 4 which can change the current or voltage control level, either with digital or analog circuitry. Further to the specific circuitry, as multiple pixels will be illuminated at any one time, each of the individual pixels is also provided with its own current source 2 so as not to divide the current between pixels and thus vary the illumination intensity according to the number of pixels which are on.

In both the passive and active cases an electronic communication protocol will be required for the circuit to communicate with the computer. This may be a wired serial interface such as USB, parallel such as GPIB-488, or wireless such as 802.11g.

### LED Cooling Structure

Cooling structures such as heat sinks and peltier cooling can be applied to the LED/control chip combination in order to reduce the temperature of GaN LED's and VCSEL's. The reduced temperature increases efficiency and thus brightness. This may not be necessary where OLED light emissive structures are incorporated.

### Casing

In some configurations, the LED illumination unit and control components may be enclosed in a humidity proof case to allow operation in humid environments such as that required for long term recording.

### Optics

Requirements. The optic system must provide the required spatial profile and sufficient irradiance for very light intensity demanding photo-stimulation processes. In addition, other factors such as appropriate working distance to accommodate for example the recording patch clamp probes, microelectrodes recording module and perfusion chambers must be taken into account. Moreover, the compatibility with the normal functions of the microscope such as fluorescent illumination and imaging must be considered.

Design consideration. LED sources have in general Lambertian emission profile (the light is emitted into a solid angle of 2π steradians and the radiant intensity is proportional to the cosine of the angle relative to the surface normal). In order to collect as much light as possible from the LED the imaging system should then have as large input NA as possible. For a small Lambertian source the collection efficiency η of a lens having a NA is given by η≈NA². For example, a lens with a NA of 0.5 will collect 25% of the total emission from the LED. Progressing through the optical projection system, the Lagrange invariant states that the brightness (power per unit area per unit solid angle) can never be increased beyond that of the object or rather that collected from the object by the input NA of the system. However, if the image is de-magnified then the output NA will be larger than the input NA (the magnification, M, is the ratio of the input NA to the output NA) and thus because the solid angle of illumination is increased the absolute irradiance (power per unit area) is then increased by 1/M². It is this irradiance (power per unit area) that is important for photo-stimulation. The trade-off in this case is that a smaller field of view is covered by the projected image and inevitably the working distance is reduced because of the use of higher NA optics.

### Method for Calibration

When the system is first put in place and later after adjustments, the total illumination of the photons hitting the sample can be measured with a calibrated photodiode. Then the variance of the intensity for each of the light spots can be measured using a professional camera with calibrated pixel responses. A computer algorithm then simply calculates the total intensity and divides by the spatial integral of the relative pixel intensities from the digital imager to find the base pixel intensity. This can be then multiplied by each individual pixel intensity to determine the exact amount of light flux passing through that point, i.e. for each light spot and hence each light source in the array. A feedback algorithm ideally controlling intensity can then be used to achieve a flat distribution of light flux over the light source array.

A similar approach can be used when the control system is arranged to intersect or combine the image of the light spots with the image of the neuron thereby to determine how much light is shining on each point on the cellular surface. This information can be used in a feedback loop with the cells such that when a desired response or action potential frequency is required at one of the cells or one point on a cell, the feedback loop will automatically adjust signalling to the light emissive electronics until the requirement is met.

### Method for Closed-Loop

The stimulus patterns and the corresponding responses of the cells recorded by the electrodes or calcium imaging are in some embodiments fed to a data processing unit that compares the performance in real time and uses it to modify in real time the stimulus. For example if a series of 10 spikes in 10 Hz is wanted from cell X, the driving circuit generates a corresponding pulse train of current, each pulse with a peak that is enough to trigger signal in the cell. Both the timing of the stimulus and the corresponding response are fed to a data-processing unit that compares the results. If in this case it found that, for example, the second and third pulse did not generate signals it will immediately send a command to increase the stimulus of the next pulse till the desired response is achieved.

The closed-loop feature can be useful for memory and plasticity studies in neurons as well as for real neuron-computer communication. Using this bilateral communication channel a neuronic chip a neuron computer or an in-vitro ‘brain’ can be developed.

### EXAMPLE

### Photo-Stimulation of Neurons

In order to use micro-LED array devices for photo-stimulation and simultaneous electrical recording of nerve cells the system shown schematically in FIG. 1 can be used. The system is constructed around an inverted microscope for sample imaging and alignment. The MEA system is mounted on the stage of this microscope and the photo-stimulation is provided by mounting the LED array and projection optics in a trans-illumination position above the stage. Ancillary electronics including LED driver and MEA instrumentation are controlled by a computer, which can adjust the stimulation and record the nerve cell response as required. Commercially available MEA recording systems typically consist of a matrix of Indium Tin Oxide electrodes on a glass substrate, coated with Titanium Nitride at the stimulation/recording points. Non-stimulating areas are passivated with silicon nitride. The array is thus fairly transparent to both trans-illuminated and epi-illuminated light. The inter-electrode spacing is typically between 100 μm and 500 μm. Typical electrode tip size of the microelectrodes is between 10 μm and 20 μm, and the electrodes are arranged in an 8×8 array.

A blue 120×1 stripe micro-LED GaN based light emitting diodes that was developed under the RCUK Basic Technology Project (V. Poher, N. Grossman et al. 2008) was used to stimulate action potentials in hippocampal neurons that were photosensitized with ChR2. The neurons were obtained from rats on embryonic day 18 and grown for 12 days in vitro. The photosensitization was achieved by transfecting the cells with ChR2. Responses from single cells were recorded with a standard patch clamping setup (HEKA epc10 double patch clamp amplifier, operating with HEKA Pulse software).

A long working distance 1:1 4F relay is based on two 50 mm triplet lenses (Sill Optics GmbH S5LPJ2851). This system although not diffraction limited, has peak to valley aberrations of less than 1.5 waves across the entire field and more than 90% of the light collected from a 17 μm diameter emitter is contained within a 32 μm diameter circle at the image plane. The working distance of this arrangement is 40 mm allowing good access to an MEA or patch-clamping.

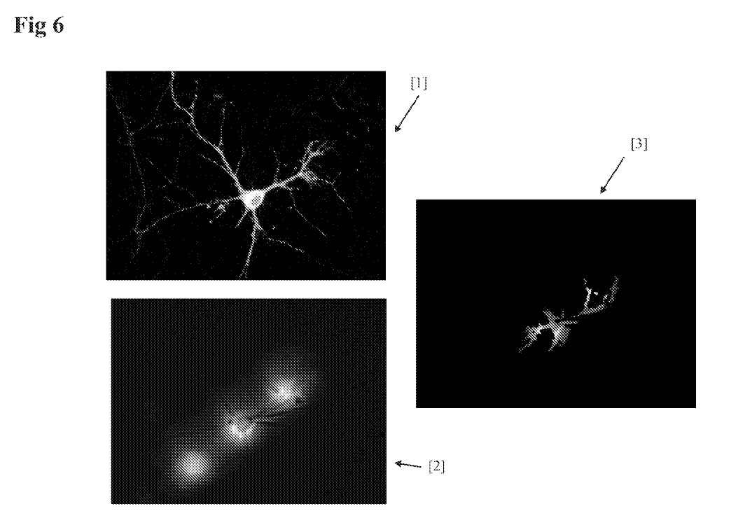

The results show a unique spatio-temporal resolution can be seen in FIG. 6 and demonstrate single cells with sub-cellular resolution. A neuron expressing GFP and ChR2 was fluorescently imaged and 3 light spots from the LED source shone on sub-cellular components. The system automatically calculated the light distribution on each part of the neuron. FIG. 6[1] shows a fluorescent image of a neuron, FIG. 6[2] shows and image of light spots from the LED, and FIG. 6[3] shows the intersection of the light spots over the neurons, which gives an exact distribution of the light intensity hitting each part of the neuron.

### REFERENCES

Banghart, M., K. Borges, et al. (2004). “Light-activated ion channels for remote control of neuronal firing.” Nature Neuroscience 7 (12): 1381-1386

Bernardinelli, Y., C. Haeberli, et al. (2005). “Flash photolysis using a light emitting diode: An efficient, compact, and affordable solution.” Cell Calcium 37 (6): 565-572.

Nikolic, K., P. Degenaar, et al. (2006). Modeling and Engineering aspects of ChannelRhodopsin2 System for Neural Photostimulation. Engineering in Medicine and Biology Society, 2006. EMBS '06. 28th Annual International Conference of the IEEE.

V. Poher, N. Grossman, et al. (2008). “Micro-LED arrays: a tool for two-dimensional neuron stimulation.” Journal of Physics D: Applied Physics 41.

Zhang, F., L.-P. Wang, et al. (2007). “Multimodal fast optical interrogation of neural circuitry.” Nature 446 (7136): 633-639.

1. A system for optical stimulation of cells, the system comprising:
an array of light sources arranged to emit light;
an optical system for directing the light from the sources to a sample location; and
a control system to control the operation of each of the sources.

2. The system according to claim 1 wherein the optical system comprises a plurality of light source lenses, each arranged to direct the light from a respective one of the light sources.

3. The system according to claim 2 wherein the array of light sources extends over an area, the light sources have gaps between them so that only a fraction of the area of the array is light-producing, and the lenses are arranged to direct the light so as to produce an image of the sources over an image area at the sample location, and so that, in the image of the sources a fraction of the image area is illuminated with light from the sources, and wherein the fraction of the image area is greater than the fraction of the area of the array.

4. The system according to claim 1 wherein the optical system comprises at least one imaging component arranged to form an image of the light sources at the sample location.

5. The system according to claim 1 wherein the optical system is arranged to cause convergence of the light from the light sources onto an area at the sample location which is smaller than the light source array.

6. The system according to claim 1 wherein the control system is arranged to control each of the light sources independently.

7. The system according to claim 6 wherein the control system is arranged to control at least one of: the intensity, the frequency of illumination pulses, and the duration of illumination pulses of the light sources.

8. The system according to claim 1 further comprising at least one sensor arranged to sense the response of cells in the sample to the stimulation.

9. The system according to claim 8 wherein the at least one sensor is arranged to output signals, and the control system is arranged to receive the signals from the at least one sensor and to control the light sources in response to the signals.

10. The system according to claim 1 further comprising an adjustable mounting on which the array of light sources is mounted.

11. The system according to claim 10 wherein the control system is arranged to adjust the adjustable mounting to adjust the position of the array.

12. The system according to claim 1 wherein the optical system comprises at least one component which is adjustable, and the control system is arranged to adjust said at least one component.

13. The system according to claim 1 further comprising a chip, wherein the light sources and light source lenses are mounted on the chip and the control system is also formed on the chip.

14. A method of calibrating a system for optical stimulation of cells, the method comprising:
providing an optical simulation system comprising an array of light sources arranged to emit light, an optical system for directing the light from the sources to a sample location, and a control system to control the operation of each of the sources;
forming an image of the light sources, the image having an illumination intensity and the illumination intensity having a variance over at least a part of the image;
measuring the total illumination in the image;
measuring the variance in illumination intensity over at least a part of the image; and
controlling the light sources to achieve a desired variance in illumination intensity over at least a part of the image.

15. The method according to claim 14 further comprising determining the absolute intensity at at least one point in the image.

16. The method according to claim 14 wherein the light sources are controlled so that they each provide the same level of illumination.

17. A microscope system comprising;
a light source arranged to illuminate a sample position;
an objective lens arranged to image the sample position;
an optical stimulation system comprising an array of light sources arranged to emit light, an optical system for directing the light from the sources to a sample location, and a control system to control the operation of each of the sources; and
a light directing system arranged to direct light from the array of light sources onto the sample position.

18. The system according to claim 17 wherein the system defines an optical path and the light directing system is arranged to introduce the light from the array of light sources into the optical path on an opposite side of the sample position to the objective lens.

19. The system according to claim 17 wherein the system defines an optical path and the light directing system is arranged to introduce the light from the array of light sources into the optical path on the same side of the sample position to the objective lens.

20. The system according to claim 1 wherein the system is formed as a modular unit arranged for connection to a port of a microscope.

21-23. (canceled)
